SEQUENCE LISTING (10 sequences)

SEQ ID NO: 1 - protein, 15 aa, synthetic construct
GNFNSSNIMKTCLQN

SEQ ID NO: 2 - protein, 18 aa, synthetic construct
GNFNSSNIMKTCLQNRGD

SEQ ID NO: 3 - protein, 18 aa, synthetic construct
NRKINMGSFTQCSNNLGD

SEQ ID NO: 4-9 - skipped (6 entries)

SEQ ID NO: 10 - protein, 9 aa, synthetic construct
CYGGRGDTP

(19) United States
(12) Patent Application Publication
(10) Pub. No.: US 2026/0159577 A1
(43) Pub. Date: Jun. 11, 2026

(54) CHIMERIC COMPOUND FOR SIMULTANEOUSLY TARGETING OF MULTIPLE DAMAGE-ASSOCIATED MOLECULAR PATTERNS (DAMPs) FOR TREATMENT OF INFLAMMATORY DISEASES

(71) Applicant: THE FEINSTEIN INSTITUTES FOR MEDICAL RESEARCH, Manhasset, NY (US)

(72) Inventors: Monowar Aziz, Manhasset, NY (US); Ping Wang, Manhasset, NY (US)

(21) Appl. No.: 19/341,141

(22) Filed: Sep. 26, 2025

Related U.S. Application Data
(60) Provisional application No. 63/701,015, filed on Sep. 30, 2024.

(51) Int. Cl. C07K 16/18; A61K 38/00; A61P 29/00; C12N 15/62; G01N 33/68

(52) U.S. Cl. CPC C07K 16/18; A61K 38/00; A61P 29/00; C12N 15/62; G01N 33/68; C07K 2319/00

(57) ABSTRACT

The present invention provides polypeptides with the ability to bind selectively to damage-associated molecular patterns (DAMPs), and fusion proteins thereof, for use in treatment of uncontrolled inflammation such as that associated with sepsis. More specifically, the present disclosure is drawn to one such fusion protein, Opsonic Peptide 18 (herein after referred to as “OP18”), and its use as a therapeutic to target and reduce the activity of DAMPs thereby providing treatment of uncontrolled inflammation including that associated with sepsis.

### CROSS-REFERENCE TO RELATED APPLICATIONS

The present application claims the benefit of and priority to U.S. Provisional Patent Application Ser. No. 63/701,015, filed on Sep. 30, 2024, the entire content of each being hereby incorporated by reference.

### GOVERNMENT SUPPORT STATEMENT

This invention was made with government support under GM129633 and GM118337 awarded by the National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Feb. 13, 2026, is named “2434-15.xml” and is 5,776 bytes in size. The sequence listing contained in this .XML file is part of the specification and is hereby incorporated by reference herein in its entirety.

### TECHNICAL FIELD

The present invention provides polypeptides with the ability to bind selectively to damage-associated molecular patterns (DAMPs), and fusion proteins thereof, for use in treatment of uncontrolled inflammation such as that associated with sepsis. More specifically, the present disclosure is drawn to one such fusion protein, Opsonic Peptide 18 (herein after referred to as “OP18”), and its use as a therapeutic to target and reduce the activity of DAMPs thereby providing treatment of uncontrolled inflammation including that associated with sepsis. The disclosure provides a method of treating inflammation mediated diseases in a subject comprising administering to the subject a therapeutically effective amount of a pharmaceutical composition comprising said fusion polypeptides, including the OP18 polypeptide, polypeptide fragments, or variants thereof.

### BACKGROUND

Uncontrolled inflammation, a critical aspect in sepsis, leads to life-threatening organ dysfunction (Aziz M, Jacob A, Yang W L, Matsuda A, Wang P: Current trends in inflammatory and immunomodulatory mediators in sepsis. J Leukoc Biol 93:329-42, 2013; Hotchkiss R S, Moldawer L L, Opal S M, Reinhart K, Turnbull I R, Vincent J L: Sepsis and septic shock. Nat Rev Dis Primers 2:16045, 2016). Sepsis affects 1.7 million Americans annually and results in 270,000 deaths, making it the third leading cause of death in US hospitals (Rhee C, Dantes R, Epstein L, Murphy D J, Seymour C W, Iwashyna T J, Kadri S S, Angus D C, Danner R L, Fiore A E, Jernigan J A, Martin G S, Septimus E, Warren D K, Karcz A, Chan C, Menchaca J T, Wang R, Gruber S, Klompas M, CDC Prevention Epicenter Program: Incidence and trends of sepsis in US hospitals using clinical vs claims data, 2009-2014. JAMA 318:1241-9, 2017). Current sepsis therapy primarily involves antibiotics and supportive care, as there are no FDA-approved drugs for sepsis (Ward P A, Fattahi F: New strategies for treatment of infectious sepsis. J Leukoc Biol 106:187-92, 2019; Vincent J L: Current sepsis therapeutics. EBioMedicine 86:104318, 2022) Monotherapy clinical trials targeting a single molecule for sepsis did not yield favorable outcomes, likely because multiple harmful molecules are involved in its pathophysiology (Cavaillon J M, Singer M, Skirecki T: Sepsis therapies: learning from 30 years of failure of translational research to propose new leads. EMBO Mol Med 12: e10128, 2020; Coopersmith C M, De Backer D, Deutschman C S, Ferrer R, Lat I, Machado F R, Martin G S, Martin-Loeches I, Nunnally M E, Antonelli M, Evans L E, Hellman J, Jog S, Kesecioglu J, Levy M M, Rhodes A: Surviving sepsis campaign: research priorities for sepsis and septic shock. Intensive Care Med 44:1400-1426, 2018). Compounding drugs (cocktail therapies) might face challenges in FDA approval due to side effects and efficacy concerns. Accordingly, there remains an unmet need for novel treatments for inflammation-mediated disorders such as sepsis.

### SUMMARY

The present invention provides polypeptides with the ability to bind selectively to damage-associated molecular patterns (DAMPs) for use in treatment of inflammation-mediated disorders such as sepsis. In an embodiment, said polypeptide binds to the eCIRP, HMGB1 and H3 DAMPs. In a specific embodiment, the present disclosure provides the polypeptide of SEQ ID NO: 1 (DAMP-binding polypeptide). Also provided are variants and fragments thereof of SEQ ID NO:1. Such variants include, for example, those that increase the stability of the polypeptide and/or binding to DAMPs.

The present disclosure further provides a fusion polypeptide comprising (i) the polypeptide of SEQ ID NO: 1 that binds to DAMPs; and (ii) a polypeptide that targets binding of the fusion protein to scavenger cells such as phagocytic macrophages that mediate clearance and degradation of said DAMPs. Such fusion proteins, referred to as opsonic polypeptides, are tagged for elimination in a subject by the process of “opsonization.”

In a non-limiting embodiment, the present disclosure is drawn to one such fusion polypeptide, Opsonic Peptide 18 (herein after referred to as “OP18”: SEQ ID NO:2), and its use as a therapeutic to target and reduce the activity of DAMPss thereby providing treatment of uncontrolled inflammation such as that associated with sepsis. The OP18 polypeptide comprises a fusion polypeptide having the amino acid of SEQ ID NO; 1 fused to an RGD (Arginine-Glycine-Aspartate) amino acid sequence that targets binding to phagocytic macrophages. Said targeting results in elimination of DAMPs in a subject. Also provided are variants and fragments thereof of SEQ ID NO:2. Such variants include those that increase the stability of the polypeptide and/or binding of DAMPs.

In one aspect, the present disclosure provides nucleic acid sequences encoding for the polypeptides of SEQ ID NO:1, SEQ ID NO: 2, as well as variants and fragments thereof. Also included are vectors and cells including said nucleic acids.

In another aspect, nanoparticles are provided comprising the DAMP-binding polypeptide of SEQ ID NO: 1 or the OP18 polypeptide of SEQ ID NO:2. Such nanoparticles can be utilized for delivery of said polypeptides to a subject for treatment of inflammation-mediated disorders, including sepsis.

In further embodiments, pharmaceutical compositions comprising the DAMP-binding polypeptide of SEQ ID NO: 1, or the OP18 polypeptide of SEQ ID NO:2, and a pharmaceutical acceptable carrier are provided. Said compositions exhibit properties for use as therapeutic agents, e.g., in the treatment of inflammation-mediated disorders such as, for example, sepsis, septic shock, organ (intestine, liver, renal, cardiac, brain/cerebral) ischemia-reperfusion (I/R) injury, trauma and hemorrhage, pneumonia, acute respiratory distress syndrome (ARDS), acute lung injury (ALI), acute kidney injury (AKI), inflammatory bowel disease (IBD), traumatic brain injury, autoimmune diseases, radiation injury and other relevant disease conditions. In addition, certain embodiments relate to pharmaceutical compositions comprising polynucleotides encoding the DAMP-binding polypeptide of SEQ ID NO: 1, or the OP18 polypeptide of SEQ ID NO: 2, vectors, and host cells comprising such polynucleotides.

The present disclosure provides methods of treating inflammation-mediated disorders in a subject comprising administering to the subject a therapeutically effective amount of a pharmaceutical composition comprising the DAMP-binding polypeptide of SEQ ID NO: 1, or the OP18 polypeptide of SEQ ID NO: 2, polypeptide fragments, or variants thereof as well as polynucleotides encoding said polypeptides. For such treatments, the administration of said polypeptides or polypeptide encoding nucleotides is designed, through their interaction with DAMPs to facilitate the elimination of said DAMPs within a subject thereby alleviating the symptoms of the inflammation-mediated disorders, including sepsis and other disease conditions as mentioned above.

In another embodiment, the DAMP-binding polypeptide of SEQ ID NO: 1, or the OP18 polypeptide of SEQ ID NO: 2, may be utilized to develop DAMP-adsorbing hemofilters for patients with sepsis-induced acute organ, e.g., kidney injury, offering a novel therapeutic approach. Hemofiltration is a renal replacement therapy which is used in the intensive care setting and can be of benefit in organ dysfunction associated with sepsis. During hemofiltration, a patient's blood is passed through a set of tubing, referred to as a filtration circuit, via a machine to a semi-permeable membrane (the filter) where waste products and water (collectively called ultrafiltrate) are removed by convection. In one aspect, the hemofilter comprises the DAMP-binding polypeptide of SEQ ID NO: 1, and/or the OP18 polypeptide of SEQ ID NO: 2, for removal of DAMPs from the treated subject's blood. Replacement fluid and blood is then returned to the patient.

In yet another embodiment, kits comprising the disclosed pharmaceutical composition for treatment of inflammation-mediated disorders are provided. Such kits contain materials useful for the treatment of inflammation-mediated disorders, such as, for example, sepsis and other related diseases. The kits may comprise one or more of the following components: a container and a label or package insert with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle).

In yet another embodiment, kits comprising the DAMP-binding polypeptide of SEQ ID NO: 1, or the OP18 polypeptide of SEQ ID NO:2, may be used to detect DAMPs in patient samples, thereby providing for the diagnosis of inflammatory diseases. For example, said kit may contain reagents for performing ELISA assays that employ the DAMP-binding polypeptide of SEQ ID NO: 1, or the OP18 polypeptide of SEQ ID NO:2, for detection of DAMPs present in a subject's sample. An increase in detectable DAMPs in the assayed sample may provide a positive diagnosis of inflammation such as sepsis in said subject. As demonstrated herein, OP18's ability to bind and capture multiple DAMPs present in the blood of septic patients provides a useful reagent for detecting multiple DAMPs simultaneously in clinical samples.

### BRIEF DESCRIPTION OF FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1. Representation of a novel opsonic peptide, OP18-mediated clearance of multiple DAMPs attenuates sepsis.

FIG. 2A-F. Opsonic Peptide 18 (OP18) binds to multiple DAMPs and αᵥβ₃-integrin with high affinity. (2A) (SEQ ID NO: 2) A 15-aa shared binding site of eCIRP, HMGB1, and histone H3 on the TLR4 extracellular region was identified by computational modeling. An RGD (Arg-Gly-Asp) motif was added to this 15-aa peptide to invent OP18. (2B-E) BIAcore assay reveals strong binding between OP18 and eCIRP, HMGB1, H3 and αᵥβ₃-integrin. (2F) OP18 binds eCIRP, HMGB1, and histone H3 in the serum of septic patients. 50 μL of serum was combined with 50 μL OP18 (10 μg/mL) and added to αᵥβ₃-integrin-coated plate. After adding primary Abs for eCIRP, HMGB1, and H3 and HRP-labeled secondary Abs, each DAMP was detected (OD at 450 nm). Fold change is shown, representing OP18's ability to detect DAMPs in human blood. N=4/group. Mean±SE, Student's t-test. *p<0.05 vs. Healthy Controls.

FIG. 3. Binding interaction between OP18 and DAMPs (in silico).

FIG. 4. Amino acid sequence of TLR4 recognizing multiple DAMPs.

FIG. 5. OP18 promotes macrophage clearance of DAMPs. RAW 264.7 cells were treated with eCIRP-FITC, HMGB 1-dye647, and H3-dye405 (1 μg/mL, each)+/−OP18 (1 μg/mL). Hoechst33342 was used for nuclear staining. After 4 h, the internalization of DAMPs was assessed by confocal microscopy. Magnification: x630. Scale bar: 10 μm. Macrophages were stained with Lysotracker for lysosomes. Intracellular localizations of DAMPs in lysosomes (whitish spots) in OP18-treated macrophages at single-cell resolution are shown as determined by confocal microscopy.

FIG. 6A-E. OP18 decreases TNFα release and corrects phagocytic dysfunction by increasing IL-10 production and restoring metabolic function. (FIG. 6A) RAW 264.7 cells were treated with eCIRP, HMGB1, H3 (referred to as DAMPs, 1 μg/mL each) with OP18 (1 g/mL) or scrambled peptide (control). After 20 h, TNFα in culture supernatants were detected by ELISA. N=6/group. (FIG. 6B) RAW 264.7 cells were treated with DAMPs (1 μg/mL each) with OP18 (1 μg/mL). After 20 h, pHrodo Green E. coli was added and phagocytosis of E. coli was assessed by flow cytometry. Mean fluorescence intensity (MFI) of E. coli phagocytosis is shown. N=5/group. (FIG. 6C) Since RAW 264.7 cells do not produce IL-10, mouse peritoneal macrophages were treated with DAMPs (1 μg/mL each) with OP18 at 1 μg/mL. After 48 h, IL-10 in supernatants was detected by ELISA. N=6/group. Mean±SE, One-way ANOVA. *p<0.05 vs. DAMPs (−)/PBS; #p<0.05 vs. DAMPs (+). (FIG. 6D) Oxygen consumption rate (OCR) and (FIG. 6E) basal and maximum respiration in RAW 264.7 cells treated with DAMPs (1 μg/mL each) and OP18 (1 μg/mL) was assessed by Agilent XFPro Seahorse assay. Real-time changes in the OCR of macrophages after treatment with oligomycin (Oligo), FCCP, and rotenone (Rot) are shown. *p<0.05 vs. PBS; #p<0.05 vs. DAMPs for both basal and Max. conditions.

FIG. 7. In Vivo experimentation demonstrating in vivo effects of OP18 inhibition of DAMPs. C57BL/6 mice were induced sepsis by cecal ligation and puncture (CLP). These mice were simultaneously treated with either OP18 (0.2 mg/kg body weight) or the same dose of scrambled peptide via intraperitoneal (i.p.) injection. After 24 h of CLP and/or OP18 and scrambled peptide injection, blood and lungs were collected to assess various parameters.

FIG. 8. OP 18 treatment decreases organ injury markers in sepsis. Scrambled peptide sequence: NRKINMGSFTQCSNNLGD (SEQ ID NO: 3). *P<0.05 vs Sham. #P<0.05 vs CLP+Scramble peptide.

FIG. 9. OP18 treatment decreases serum cytokines in sepsis. Scrambled peptide sequence: NRKINMGSFTQCSNNLGD (SEQ ID NO: 3). *P<0.05 vs Sham. #P<0.05 vs CLP+Scramble peptide.

FIG. 10. OP18 treatment decreases IL-6, TNF-α, and IL-1β lung cytokine mRNA in sepsis. Scrambled peptide sequence: NRKINMGSFTQCSNNLGD (SEQ ID NO: 3). *P<0.05 vs Sham. #P<0.05 vs CLP+Scramble peptide.

FIG. 11. OP18 treatment decreases lung KC and MIP-2 chemokine mRNA. Scrambled peptide sequence: NRKINMGSFTQCSNNLGD (SEQ ID NO: 3). *P<0.05 vs Sham. #P<0.05 vs CLP+Scramble peptide.

FIG. 12. OP18 treatment decreases lung myeloperoxidase (MPO) levels. Scrambled peptide sequence: NRKINMGSFTQCSNNLGD (SEQ ID NO: 3). *P<0.05 vs Sham. #P<0.05 vs CLP+Scramble peptide.

FIG. 13. OP18 treatment decreases lung injury scores in sepsis. Scrambled peptide sequence: NRKINMGSFTQCSNNLGD (SEQ ID NO: 3).

FIG. 14. OP18 treatment decreases lung cell's apoptosis in sepsis. Scrambled peptide sequence: NRKINMGSFTQCSNNLGD (SEQ ID NO: 3).

FIG. 15. OP18 treatment improves survival in sepsis. 7-day survival study of CLP septic mice with i.p. injection of a vehicle or 0.1 mg/kg OP18 at the end of the procedure. N=25 mice/group. Survival rates were analyzed by the Kaplan-Meier estimator using a log-rank test. *p<0.05 versus vehicle.

### DETAILED DESCRIPTION

### Definitions

The term “inflammation” as used herein means a condition or disease characterized by an overactive immune system, where the body attacks and damages its own tissues and organs.

The terms “effective amount” or “therapeutically effective amount” as used herein have the standard meanings known in the art and are used interchangeably herein to mean an amount sufficient to treat a subject afflicted with a condition or disease (e.g., inflammation-mediated disorders) or to halt the progression of the condition or disease or alleviate a symptom or a complication associated with the condition or disease. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999); Fuhrman L C, Jr. Ansel's Pharmaceutical Dosage Forms and Drug Delivery Systems, 8th Edition: Am J Pharm Educ. 2006 Jun. 15; 70(3):71). For example, in the case of an agent to treat inflammation-mediated disorders, such as sepsis, an effective amount may be an amount sufficient to result in clinical improvement of the patient.

The terms “protein” “peptide” and “polypeptide” as used herein are used interchangeably, unless specified to the contrary, and according to conventional meaning, mean a sequence of amino acids. Peptides are not limited to a specific length, e.g., they may comprise a polypeptide sequence, full-length protein sequence or a fragment of a full-length protein, and may include post-translational modifications of the polypeptide, for example, glycosylations, acetylations, phosphorylations and the like, as well as other modifications known in the art, both naturally occurring and non-naturally occurring, e.g. variants.

The term “nucleic acid” as used herein refers to deoxyribonucleic acid (DNA), ribonucleic acid (RNA), cDNA, RNA and modified nucleic acids that can, for example, increase stability.

The term “subject” as used herein refers to an animal. Typically, the animal is a mammal. A subject also refers to, for example, primates (e.g., humans), cows, sheep, goats, horses, dogs, cats, rabbits, rats, mice, fish, birds and the like. In certain embodiments, the subject is a primate. In yet other embodiments, the subject is a human. A subject in need is a subject that is suffering from an inflammation mediated disorder. In a non-limiting example, the disorder is sepsis.

The term “therapeutic agent” as used herein is a compound capable of producing a desired and beneficial effect. The terms “treat,” “treating” or “treatment” of any disease or disorder as used herein refer in one embodiment, to halting the progression of the condition or disease, or to ameliorating the disease or disorder (i.e., slowing or arresting or reducing the development of the disease or at least one of the clinical symptoms thereof). In another embodiment “treat,” “treating” or “treatment” refers to alleviating or ameliorating at least one physical parameter including those which may not be discernible by the patient. In yet another embodiment, “treat,” “treating” or “treatment” refers to modulating the disease or disorder, either physically, (e.g., stabilization of a discernible symptom), physiologically, (e.g., stabilization of a physical parameter), or both. In yet another embodiment, “treat,” “treating” or “treatment” refers to preventing or delaying the onset or development or progression of the disease or disorder. As used herein, a subject is “in need of” a treatment if such subject would benefit biologically, medically or in quality of life from such treatment.

The present disclosure provides novel polypeptides capable of targeting multiple harmful molecules (DAMPs) to alleviate inflammatory-mediated disorders, such as sepsis. For example, during sepsis, stressed cells release intracellular components that, when outside the cells, transform into harmful mediators known as damage-associated molecular patterns (DAMPs). Such DAMPs include, for example, those that originate from the nucleus, including extracellular cold-inducible RNA-binding protein (eCIRP), high mobility group box-1 (HMGB1), and histones such as H3. These nuclear DAMPs bind to Toll-like receptor 4 (TLR4), amplifying inflammation and contributing to sepsis.

Accordingly, the present disclosure provides polypeptides with the ability to bind selectively to DAMPss for use in treatment of inflammation such as that associated with sepsis. In an embodiment, said polypeptide binds to the eCIRP, HMGB1 and H3 DAMP proteins and comprises the amino acid sequence: GNFNSSNIMKTCLQN (“DAMP-binding polypeptide”; SEQ ID NO: 1). DAMP-binding polypeptides also include polypeptide fragments of said polypeptide, as well as variants of the polypeptide. Full length polypeptide, polypeptide fragments and variants are collectively referred to herein in “DAMP-binding polypeptides.”

In another embodiment, the present disclosure provides a fusion protein comprising the amino acid sequence of SEQ ID NO: 1 fused to a second polypeptide that directs the binding of the fusion protein to a scavenger cell. Such scavenger cells include, for example, macrophages and endothelial scavenger cells that actively seek out and engulf disease-causing proteins, pathogens, and infected cells. Such scavenger cells express a remarkably high blood clearance activity and therefore find use in the clearance of DAMPs from a subject. Scavenger cell receptors include, for example, those expressed on the surface of phagocytic macrophages and scavenger endothelial cells. In a specific embodiment, the polypeptide directing binding to phagocytic macrophages is the tripeptide RGD. Additional scavenger cells, other than macrophage/monocytes or scavenger endothelial cells include, but are not limited to, neutrophils, dendritic cells, B lymphocytes (B-1 and B-2 cells), Kupffer cells, glial cells, and keratinocytes. Given the recognition of the RGD motif by other integrins, including αᵥβ₅ and αᵥβ₁, these integrins may also function as scavenging receptors contributing to OP18-mediated clearance of DAMPs. In addition to the RGD motif, other scavenger motifs may be used to direct the binding of the fusion protein to a scavenger cell. Such scavenger cell motifs include, for example, those that bind to MARCO which serves as a scavenger receptor for apoptotic cells. Therefore, any ligand that binds to MARCO, can be used to tag fusion proteins for facilitating MARCO mediated clearance.

Accordingly, the present disclosure further provides a fusion polypeptide comprising (i) the polypeptide of SEQ ID NO: 1 that binds to DAMPs; and (ii) a polypeptide that targets binding to scavenger cells, such as phagocytic macrophages, thereby mediating clearance and degradation of said DAMPs within a subject. Such fusion proteins, referred to as opsonic polypeptides, are tagged for elimination in a subject by the process of “opsonization.”

In a specific embodiment, the present disclosure provides an 18-amino acid fusion polypeptide, referred to as Opsonic Peptide 18 (OP18) that comprises (i) a region of the TLR4 receptor that is able to bind to multiple DAMPs (i.e., SEQ ID NO:1) and (ii) an αᵥβ₃-integrin binding RGD (Arg-Gly-Asp) motif that targets binding to phagocytic macrophages thereby mediating clearance and degradation of multiple DAMPs within a subject. Accordingly, the present disclosure provides methods for inhibiting the activity of DAMPs in a subject comprising administering to the subject an effective amount of OP18 in a pharmaceutically acceptable form. As used herein, the term “OP18” refers to a polypeptide having the following amino acid sequence (SEQ ID NO: 2): (GNFNSSNIMKTCLQNRGD). In one aspect, nucleic acid sequences encoding the OP18 polypeptide of SEQ ID NO:2 are provided herein.

Such OP18 polypeptides also include polypeptide fragments of OP18, as well as variants of the polypeptide. Full length polypeptide, polypeptide fragments and variants are collectively referred to herein in “OP18 polypeptides”.

The skilled artisan will readily appreciate that the embodiments are not limited to the DAMP-binding polypeptide and OP18 polypeptide amino acid sequences depicted herein but also include variants of said polypeptides. Such variants may contain deletions, substitutions, or additions of one or more amino acids in the above depicted amino acid sequence of SEQ ID NO: 1, or SEQ ID NO:2, while maintaining the biological activity of said polypeptides. Such variants include those, for example, that increase the half-life or stability of the OP18 polypeptide or increase the affinity and binding of said polypeptides to DAMPs. Such fragments or variants may be naturally occurring or may be synthetically generated, for example, by modifying one or more of the above polypeptide sequences of SEQ ID NO: 1 or SEQ ID NO:2 used in the methods of certain embodiments and evaluating their effects using any of a number of techniques well known in the art. In a specific embodiment, 1, 2, 3, 4, or 5 amino acids of DAMP-binding polypeptide or the OP18 polypeptide are substituted resulting in analogs and/or derivatives of said polypeptides.

As used herein, a peptide fragment or variant has amino acid sequences that share at least about 70-75%, typically at least about 80-85%, and most typically at least about 90-95%, 97%, 98% or 99% or more identity with the DAMP-binding polypeptide of SEQ ID NO: 1 or the OP18 polypeptide of SEQ ID NO. 2 or peptide fragments thereof. Modifications may be made in the structure of polypeptides of certain embodiments and still obtain a functional molecule that encodes a variant or derivative polypeptide with desirable characteristics, e.g. binding to DAMPs and/or scavenger cells.

The term “identity” or “sequence identity” is known in the art and refers to a relationship between two or more polynucleotide or amino acid sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48:1073 (1988), the teachings of which are incorporated herein by reference. Methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference).

In certain embodiments, a fragment or variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Suitable conservative substitutions of amino acids are known to those of skill in this art and generally can be made without altering the biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). One of skill in the art could determine which amino acid residues can be substituted, inserted, or deleted without abolishing biological activity. Assistance can be found using computer programs well known in the art, such as DNASTAR™ software. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

Fragments, or variants, or derivatives of the DAMP-binding polypeptide or the OP18 polypeptide include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and mutants. Truncations or deletions of regions which change functional activity of the proteins are also variants.

DAMP-binding polypeptides, or OP18 polypeptides, also include polypeptides wherein the polypeptide has been modified for the purpose of improving bioavailability, and/or increasing efficacy, solubility and stability of such polypeptides. Such modifications included, for example, the addition of a C-terminal amide group and/or a N-terminal acetyl group. The polypeptides, for use as disclosed herein, may be chemically modified. For example, polypeptides may be covalently or non-covalently linked to albumin, transferrin or additional polyethylene glycol (PEG) moieties. In some embodiments, the polypeptides can be modified so that they have an extended half-life in vivo using any methods known in the art. In an embodiment, peptidomimetics of the DAMP-binding polypeptide, or OP18 polypeptide, may be generated to decrease the susceptibility towards protease cleavage. For example, a number of different unnatural amino acids may be utilized in the synthesis of polypeptides. For example, a mirror-image (D-peptide) of the DAMP-binding polypeptide or OP18 polypeptide may be produced to increase the stability of the polypeptides. Cyclization of peptides has been shown to enhance stability against proteolytic degradation and accordingly can be used to enhance stability of the polypeptides of interest. This is commonly done by linking the C-terminus to the N-terminus, but alternative methods include linking to side chains. Cyclic peptides can be arranged in various ways, such as head-to-tail, head-to-side chain, tail-to-side chain, and side chain-to-side chain. In yet another embodiment, to enhance the metabolic stability of OP18 peptide, amide groups in the peptide backbone can be replaced with more stable sulfonamide groups such as those commonly used in sulfa drugs.

The present disclosure further provides, nucleic acid molecules encoding the DAMP-binding polypeptide of SEQ ID NO:1 or the OP18 polypeptide of SEQ ID NO:2. Due to the degeneracy of nucleotide coding sequences, a variety of nucleic acid sequences which encode the same or a substantially similar amino acid sequences of said polypeptides may be used in the practice of the present disclosure to prepare expression vectors for the production of recombinant the DAMP-binding polypeptide of SEQ ID NO: 1 and the OP18 polypeptide of SEQ ID NO:2, including said polypeptides variants. These include, but are not limited to, nucleic acid molecules encoding all or portions of the DAMP-binding polypeptide of SEQ ID NO:1, or the OP18 polypeptide of SEQ ID NO:2, including those that are altered by the substitution of different codons that encode the same or a functionally equivalent amino acid residue within the sequence, thus producing a silent change. In one aspect, the substitution of different codons may be used to optimize the codon usage depending on the species used for expression of the polypeptides.

In one aspect, the provided nucleic acid molecules encode a peptide fragment or variant of the DAMP-binding polypeptide of SEQ ID NO: 1, or the OP18 polypeptide of SEQ ID NO:2, that has amino acid sequences that are at least about 70-75%, typically at least about 80-85%, and most typically at least about 90-95%, 97%, 98% or 99% or more identical with the DAMP-binding polypeptide of SEQ ID NO: 1 or the OP18 polypeptide of SEQ ID NO. 2 or peptide fragments thereof.

The DAMP-binding polypeptide of SEQ ID NO:1, or the OP18 polypeptide of SEQ ID NO: 2, polypeptide fragments or variants thereof, for use in the methods disclosed herein may be made in a variety of ways. For example, solid phase synthesis techniques may be used. Suitable techniques are well known in the art, and include those described in Merrifield, in Chem. Polypeptides, pp. 335-61 (Katsoyannis and Panayotis eds. 1973); Merrifield, J. Am. Chem. Soc. 85:2149 (1963); Davis et al., Biochem. Intl. 10:394-414 (1985); Stewart and Young, Solid Phase Peptide Synthesis (1969); U.S. Pat. No. 3,941,763; Finn et al., The Proteins, 3rd ed., vol. 2, pp. 105-253 (1976); and Erickson et al., The Proteins, 3rd ed., vol. 2, pp. 257-527 (1976).

The short amino acid sequences of the DAMP-binding polypeptide of SEQ ID NO:1, or the OP18 polypeptide of SEQ ID NO:2, make synthetic production of these valuable polypeptides readily practicable, and a variety of automated instruments for peptide synthesis are commercially available, and synthetic methods for peptide synthesis have long been known and can be used in accordance with the teachings herein to prepare the DAMP-binding polypeptide or the OP18 polypeptide.

Purification of the resulting DAMP-binding polypeptide, or the OP18 polypeptide is accomplished using conventional procedures. Any techniques known in the art can be used in purifying of the DAMP-binding polypeptide of SEQ ID NO: 1 or the OP18 polypeptide of SEQ ID NO: 2, including, but not limited to, purification by precipitation, adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or gel filtration, electrophoresis, liquid phase partitioning, detergent partitioning, organic solvent extraction, and ultrafiltration. During purification, the biological activity of the DAMP-binding polypeptide, e.g., DAMP-binding, or the OP18 polypeptide, e.g. DAMP binding and scavenger cell binding, may be monitored by one or more in vitro or in vivo assays. The purity of DAMP-binding polypeptide, or the OP18 polypeptide, can be assayed by any methods known in the art, such as but not limited to, gel electrophoresis or HPLC.

In addition to synthetic chemistry methods, DAMP-binding polypeptides, or OP18 polypeptides, for use as disclosed herein can be produced by standard recombinant DNA techniques in accordance with the teachings herein and known in the art. For example, nucleic acid molecules coding for the different polypeptide sequences may be ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Furthermore, the nucleic acid molecules can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence.

Methods known in the art can be utilized to produce the polypeptides disclosed herein, including the DAMP-binding polypeptide, or the OP18 polypeptide, recombinantly. A nucleic acid sequence encoding said polypeptides, can be inserted into an expression vector for propagation and expression in host cells. The term “expression vector” as used herein refers to any molecule or entity (e.g., nucleic acid, plasmid, bacteriophage or virus) used to transfer protein coding information into a host cell. An “expression vector” or “expression construct” as used herein refers to a recombinant DNA molecule containing a desired coding sequence and appropriate nucleic acid control sequences necessary for the expression of the operably linked coding sequence in a particular host cell. Vectors include viral vectors such as adenovirus, Herpes simplex I virus, adeno-associated virus, lentivirus or retrovirus vectors.

Accordingly, certain embodiments include a vector encoding DAMP-binding polypeptides, or OP18 polypeptides, in an appropriate host. The vector comprises the DNA molecule that encodes DAMP-binding polypeptides, or OP18 polypeptides, operatively linked to appropriate expression control sequences. Methods of affecting this operative linking are well known. Expression control sequences include promoters, activators, enhancers, operators, ribosomal binding sites, start signals, stop signals, cap signals, polyadenylation signals, and other signals involved with the control of transcription or translation. In an embodiment, vectors include viral vectors such as adenovirus, Herpes simplex I virus, adeno-associated virus, lentivirus or retrovirus vectors.

Methods are well known to one of skill in the art and can be used to construct expression vectors containing the coding sequence with appropriate transcriptional and translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989).

Moreover, expression vectors commercially available that already encode a fusion moiety may be used to express a DAMP-binding fusion polypeptide, or an OP18 fusion polypeptide. A DAMP-binding polypeptide encoding nucleic acid, or an OP18 polypeptide-encoding nucleic acid, can be cloned into such an expression vector such that the fusion moiety is linked in-frame to the DAMP binding polypeptide, or OP18 polypeptide, encoding nucleic acid. The fusion protein can be a DAMP-binding polypeptide, or an OP18 polypeptide fused to a His tag or epitope tag (e.g. V5) to aid in the purification and detection of the recombinant polypeptides.

The resulting expression vector comprising the DAMP-binding polypeptide, or OP18 polypeptide, encoding nucleic acid molecule is used to transform an appropriate host cell. This transformation may be performed using methods well known in the art. Any of a large number of available and well-known host cells may be used in the practice of these embodiments. The selection of a particular host cell is dependent upon a number of factors recognized by the art. These factors include, for example, compatibility with the chosen expression vector, toxicity to the host cell of the proteins encoded by the DNA molecule, rate of transformation, ease of recovery of the proteins, expression characteristics, biosafety and costs. A balance of these factors must be struck with the understanding that not all hosts may be equally effective for the expression of a particular DNA sequence.

A variety of techniques are available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, chemical treatments, DEAE-dextran, and calcium phosphate precipitation. Other in vivo nucleic acid transfer techniques include transfection with viral vectors (such as adenovirus, Herpes simplex I virus, adeno-associated virus, lentivirus or retrovirus) and lipid-based systems. The nucleic acid and transfection agent are optionally associated with a microparticle.

Exemplary transfection agents include calcium phosphate or calcium chloride co-precipitation, DEAE-dextran-mediated transfection, quaternary ammonium amphiphile DOTMA ((dioleoyloxypropyl)trimethylammonium bromide, commercialized as Lipofectin by GIBCO-BRL)) (Felgner et al, (1987) Proc. Natl. Acad. Sci. USA 84, 7413-7417; Malone et al. (1989) Proc. Natl Acad. Sci. USA 86 6077-6081); lipophilic glutamate diesters with pendent trimethylammonium heads (Ito et al. (1990) Biochem. Biophys. Acta 1023, 124-132); the metabolizable parent lipids such as the cationic lipid dioctadecylamido glycylspermine (DOGS, Transfectam, Promega) and dipalmitoylphosphatidyl ethanolamylspermine (DPPES) (J. P. Behr (1986) Tetrahedron Lett. 27, 5861-5864; J. P. Behr et al. (1989) Proc. Natl. Acad. Sci. USA 86, 6982-6986); metabolizable quaternary ammonium salts (DOTB, N-(1-[2,3-dioleoyloxy]propyl)-N,N,N-trimethylammonium methylsulfate (DOTAP) (Boehringer Mannheim), polyethyleneimine (PEI), dioleoyl esters, ChoTB, ChoSC, DOSC) (Leventis et al. (1990) Biochim. Inter. 22, 235-241); 3 beta [N—(N′,N′-dimethylaminoethane)-carbamoyl] cholesterol (DC-Chol), dioleoylphosphatidyl ethanolamine (DOPE)/3beta [N—(N′,N′-dimethylaminoethane)-carbamoyl] cholesterolDC-Chol in one to one mixtures (Gao et al., (1991) Biochim. Biophys. Acta 1065, 8-14), spermine, spermidine, lipopolyamines (Behr et al., Bioconjugate Chem, 1994, 5:382-389), lipophilic polylysines (LPLL) (Zhou et al., (1991) Biochim. Biophys. Acta 939, 8-18), [[(1, 1, 3, 3-tetramethylbutyl)cre-soxy] ethoxy] ethyl] dimethylbenzylammonium hydroxide (DEBDA hydroxide) with excess phosphatidylcholine/cholesterol (Ballas et al., (1988) Biochim. Biophys. Acta 939, 8-18), cetyltrimethylammonium bromide (CTAB)/DOPE mixtures (Pinnaduwage et al, (1989) Biochim. Biophys. Acta 985, 33-37), lipophilic diester of glutamic acid (TMAG) with DOPE, CTAB, DEBDA, didodecylammonium bromide (DDAB), and stearylamine in admixture with phosphatidylethanolamine (Rose et al., (1991) Biotechnique 10, 520-525), DDAB/DOPE (TransfectACE, GIBCO BRL), and oligogalactose bearing lipids. Exemplary transfection enhancer agents that increase the efficiency of transfer include, for example, DEAE-dextran, polybrene, lysosome-disruptive peptide (Ohmori N I et al, Biochem Biophys Res Commun Jun. 27, 1997; 235(3):726-9), chondroitan-based proteoglycans, sulfated proteoglycans, polyethylenimine, polylysine (Pollard H et al. J Biol Chem, 1998 273 (13): 7507-11), integrin-binding peptide CYGGRGDTP (SEQ ID NO: 10), linear dextran nonasaccharide, glycerol, cholesteryl groups tethered at the 3′-terminal internucleoside link of an oligonucleotide (Letsinger, R. L. 1989 Proc Natl Acad Sci USA 86: (17):6553-6), lysophosphatide, lysophosphatidylcholine, lysophosphatidylethanolamine, and 1-oleo yl lysophosphatidylcholine.

The host cell can be prokaryotic (bacteria) or eukaryotic (e.g., yeast, insect, plant and animal cells). In a preferred embodiment the host cell is a mammalian cell. Exemplary mammalian host cells are COS1 and COS7 cells, NSO cells, Chinese hamster ovary (CHO) cells, NIH3T3 cells, HEK293 cells, HEPG2 cells, HeLa cells, L cells, MDCK, W138, murine ES cell lines (e.g., from strains 129/SV, C57/BL6, DBA-1, 129/SVJ), K562, Jurkat cells, BW5147 and any other commercially available human cell lines. Other useful mammalian cell lines are well known and readily available from the American Type Culture Collection (ATCC) (Manassas, Va., USA) and the National Institute of General Medical Sciences (NIGMS) Human Genetic Cell Repository at the Coriell Cell Repositories (Camden, N.J., USA).

Next, the transformed host is cultured under conditions so that the desired DAMP-binding polypeptide, or OP18 polypeptide, is expressed. Such conditions are well known in the art. Finally, the desired polypeptides are purified from the fermentation culture or from the host cells in which they are expressed. These purification methods are also well known in the art. For example, DAMP-binding polypeptides, or OP18 polypeptides, prepared as described herein may be purified by art-known techniques such as high-performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification, an antibody, ligand, receptor or antigen can be used to which the DAMP-binding polypeptide or OP18 polypeptide, binds. In addition, size exclusion chromatography can be used to isolate DAMP-binding polypeptides or OP18 polypeptides.

The present disclosure further provides DAMP-binding polypeptide, or OP18 polypeptide, containing nanoparticles and methods of using and making thereof. More specifically, the provided nanoparticles comprise an outer surface comprising DAMP-binding polypeptides or OP18 polypeptides. Said nanoparticles are designed to bind to DAMP proteins and mediate clearance from the subject. Said nanoparticle may also target binding to a scavenger cell for clearance from the subject. In certain embodiments, the inner core of the inventive nanoparticle comprises a biocompatible and/or a synthetic material. In a specific embodiment, the nanoparticle is formed from a biocompatible polymer. In a specific embodiment the nanoparticle is a nanoliposome.

The present disclosure further provides a method for making the DAMP-binding polypeptide, or OP18 polypeptide, containing nanoparticles disclosed herein. Such methods, well known those those of skill in the art include, for example, chemical reduction, coprecipitation, seeding, microemulsion, inverse microemulsion, hydrothermal method, and sonic deposition. For coating with DAMP-binding polypeptides, OP18 polypeptides, or scavenger cell targeting polypeptides, the nanoparticles may be incubated with a solution comprising said polypeptides. In one aspect, the nanoparticles are designed for efficient and favorable renal and/or hepatic clearance.

The term “nanoparticle” as used herein refers to nanostructure, particles, vesicles, or fragments thereof having at least one dimension (e.g., height, length, width, or diameter) of between about 1 nm and about 10 pm. For systemic use, an average diameter of about 50 nm to about 500 nm, or 100 nm to 250 nm may be preferred. In certain embodiments, the nanoparticles provided herein are biocompatible and/or biodegradable. The nanoparticles can be composed of organic materials or other materials and can alternatively be implemented with porous particles. The layer of nanoparticles can be implemented with nanoparticles in a monolayer or with a layer having agglomerations of nanoparticles. As used herein, the nanoparticle consists of a DAMP-binding polypeptide, and/or an OP18 polypeptide.

Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof.

In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy(polyethylene glycol)-2000](DPPE-PEG2000), 1,2-distearoyl-sn-glycero-3-phosphoethanolamine-N-[methoxy(polyethylene glycol)-1000] (DSPE-PEG2000), and cholesterol.

The present disclosure further provides that the nanoparticle, having a DAMP-binding polypeptide, or OP18 polypeptide coating, comprises a payload that can be located in any place inside or on the surface of the nanoparticle. In certain embodiments, the payload comprises one or more therapeutic agents, prophylactic agents, diagnostic agents, or a combination thereof. Examples of therapeutic agents include, but are not limited to, an antibiotic, an antimicrobial, a growth factor, a chemotherapeutic agent, an anti-inflamatory, or a combination thereof. In a specific embodiment, the payload comprises one or more therapeutic agents for use in the treatment of inflammation-mediated disorders such as sepsis.

Crosslinking agents suitable for crosslinking the proteins to produce the nanoparticle, or to coat the proteins on the nanoparticle are known in the art, and include those selected from the group consisting of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfhydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, and Sulfo-EGS.

In further embodiments, pharmaceutical compositions comprising the DAMP-binding polypeptide, or OP18 polypeptide, and a pharmaceutical acceptable carrier are provided. Said pharmaceutical compositions exhibit properties for use as a therapeutic agent, e.g., in the treatment of inflammation-mediated disorders, including sepsis. In addition, certain embodiments relate to compositions comprising nucleic acid molecules encoding the DAMP-binding polypeptides, or OP18 polypeptides, vectors, and host cells comprising such polypeptides. In yet another embodiment, kits comprising said pharmaceutical compositions are provided. In another embodiment, a pharmaceutical composition comprises any of the DAMP-binding polypeptides, or OP18 polypeptides provided herein and at least one additional therapeutic agent, typically used for treatment of inflammation-mediated disorders, including sepsis are provided.

Pharmaceutical compositions of embodiments comprise a therapeutically effective amount of one or more DAMP-binding polypeptides, or OP18 polypeptides, dissolved or dispersed in a pharmaceutically acceptable carrier. The preparation of said pharmaceutical compositions are well known to those of skill in the art as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in therapeutic or pharmaceutical compositions is contemplated.

The composition may comprise different types of carriers depending on whether it is to be administered in solid, liquid or aerosol form, and whether it needs to be sterile for such routes of administration as injection. DAMP-binding polypeptides, or OP18 polypeptide, of certain embodiments (and any additional therapeutic agent) can be administered by any method, or any combination of methods as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering protein or polypeptide molecules such as the DAMP-binding polypeptides, or OP18 polypeptides, of certain embodiments. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Parenteral compositions include those designed for administration by injection, e.g. subcutaneous, intradermal, intra-lesional, intravenous, intra-arterial, intramuscular, intrathecal, intratracheal, intraperitoneal, and intrarectal injection/administration. For injection/delivery, the DAMP-binding polypeptides, or OP18 polypeptides, may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the DAMP-binding polypeptides, or OP18 polypeptides, may be in powder form for constitution with a suitable vehicle, e.g., sterile pyrogen-free water, before use. Sterile injectable solutions are prepared by incorporating the OP18 polypeptide in the required amount in the appropriate solvent with various other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi.

Pharmaceutical compositions comprising DAMP-binding polypeptides, or OP18 polypeptides, may be manufactured by means of conventional mixing, dissolving, emulsifying, encapsulating, entrapping or lyophilizing processes. Pharmaceutical compositions may be formulated in conventional manners using one or more physiologically acceptable carriers, diluents, excipients or auxiliaries which facilitate processing of the proteins into preparations that can be used pharmaceutically. Proper formulation is dependent upon the route of administration chosen.

DAMP-binding polypeptides, or OP18 polypeptides, may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as, for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The pharmaceutical preparation of certain embodiments is a liquid composition, e.g. an aqueous solution. For injection purposes, the use of pure water as solvent is preferred. Other solvents which are suitable and conventional for pharmaceutical preparations can, however, also be employed. In a preferred embodiment, the pharmaceutical compositions are isotonic solutions. Further, there is no need for reconstitution at any stage of the preparation of the liquid solution formulation of these embodiments. The solution is a ready-to-use formulation.

The delivery of therapeutic DAMP-binding polypeptides, or OP18 polypeptides, within a host can occur via gene therapy ex vivo, in situ, or in vivo by use of any suitable approach known in the art. For example, for in vivo therapy, a nucleic acid encoding the desired DAMP-binding polypeptides, or OP18 polypeptides, either alone or in conjunction with a vector, liposome, or precipitate may be injected directly into the subject, and in some embodiments, may be injected at the site where the expression of the polypeptides is desired. For ex vivo treatment, the subject's cells are removed, the nucleic acid is introduced into these cells, and the modified cells are returned to the subject either directly or, for example, encapsulated within porous membranes which are implanted into the patient. See, e.g. U.S. Pat. Nos. 4,892,538 and 5,283,187.

The present disclosure provides a method of treating a subject suffering from an inflammation-mediated disease, or at-risk factor for developing an inflammation-mediated disease, the method comprising administering to the subject, an effective amount of DAMP-binding polypeptides, or OP18 polypeptides in a pharmaceutically acceptable form.

Such inflammation-mediated disorders but may be selected from the group consisting of inflammatory lung disease, inflammatory liver disease, cardiac disease, inflammatory bowel disease, autoimmune disease, inflammatory central nervous system disease, inflammatory skin disease, and allergic inflammatory disease. More specifically, the inflammatory disease may be selected from the group consisting of interstitial lung disease (ILD), non-alcoholic steatohepatitis (NASH), Crohn's disease, ulcerative colitis, rheumatoid arthritis, type 1 diabetes, lupus, multiple sclerosis, Parkinson's disease, scleroderma, psoriasis, lupus, psoriasis, and ankylosing spondylitis, heart disease, ulcerative colitis, irritable bowel syndrome, asthma, chronic obstructive pulmonary disease (COPD), Type 2 diabetes, Alzheimer's disease, and Parkinson's disease. In some instances, inflammation is associated with a higher risk of cancer. Such an inflammation-mediated disorders include, for example, those resulting from release af various DAMPs.

One particular example of inflammation is sepsis that occurs when your body's immune system has a severe reaction to an infection, causing inflammation throughout your body. This inflammation can lead to tissue damage, organ failure, and even death. Sepsis occurs when your immune system has a dangerous reaction to an infection. It causes extensive inflammation throughout your body that can lead to tissue damage, organ failure and even death.

Such, “inflammation” means that the subject has symptoms typically associated with inflammation. Such symptoms may be attributable to the presence in the subject of an inflammatory disease or injury to the subject. Such inflammation symptoms include, for example, fever, chills, fatigue/loss of energy, headaches, loss of appetite, muscle stiffness, redness and swelling at site of injury.

Any of the DAMP-binding peptides, OP18 polypeptides, or polypeptide encoding nucleic acids, provided herein may be used in therapeutic methods described herein. For use in the therapeutic methods described herein, such polypeptides, or encoding nucleic acids of certain embodiments would be formulated, dosed, and administered in a fashion consistent with good medical practice.

For the treatment of inflammation-mediated disorders, the appropriate dosage of DAMP-binding polypeptide, or OP18 polypeptide (when used alone or in combination with one or more other additional therapeutic agents) will depend on the type of disease to be treated, the route of administration, the body weight of the patient, the severity and course of the disease, whether the protein is administered for preventive or therapeutic purposes, previous or concurrent therapeutic interventions, the patient's clinical history and response to the DAMP-binding polypeptide, or OP18 polypeptide, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The DAMP-binding polypeptides, or OP18 polypeptides, are suitably administered to the patient at one time or over a series of treatments subcutaneously, intravenously, intramuscularly, locally or via airway or under tongue. For repeated administrations over several days or longer, depending on the condition, the treatment would generally be sustained until a desired suppression of disease symptoms occurs.

One typical dosage would be in the range from about 1 μg/kg body weight to 1000 mg/kg body weight. In other non-limiting examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein.

Such doses may be administered intermittently, e.g., every week or every three weeks. An initial higher loading dose, followed by one or more lower doses may be administered. However, other dosage regimens may be useful. The progress of this therapy is easily monitored by conventional techniques and assays. The DAMP-binding polypeptides, or OP18 polypeptides of certain embodiments will generally be used in an amount effective to achieve the intended purpose. For use to treat or prevent inflammation such as sepsis, the DAMP-binding polypeptides, or OP18 polypeptides, of these embodiments, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data. In a non-limiting embodiment, a mouse dose in mg/kg may be converted to a human equivalent dose (HED) in mg/kg, by multiplying the mouse dose by 0.081 (PMC4804402). In a non-limiting embodiment, it has been found that OP18 has been effective in mouse sepsis at doses as low as 0.1 mg/kg and 0.2 mg/kg.

The OP18 containing compositions may be administered by an initial bolus followed by a continuous infusion to maintain therapeutic circulating levels of drug product. As another example, the inventive compound may be administered as a one-time dose. Those of ordinary skill in the art will readily optimize effective dosages and administration regimens as determined by good medical practice and the clinical condition of the individual patient.

The attending physician for patients treated with DAMP-binding polypeptides, or OP18 polypeptide of certain embodiments would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The DAMP-binding polypeptides, or OP18 polypeptides, described herein may be administered in combination with one or more other agents or “therapeutic agents” for use in treatment of an inflammation-mediated diseases. The polypeptides may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other.

In another embodiment, the DAMP-binding polypeptide of SEQ ID NO: 1, or the OP18 polypeptide of SEQ ID NO: 2 may be utilized to develop DAMP-adsorbing hemofilters for patients with sepsis-induced acute kidney injury, offering a novel therapeutic approach. Hemofiltration is a renal replacement therapy which is used in the intensive care setting and can be of benefit in organ dysfunction associated with inflammation such as sepsis. During hemofiltration, a patient's blood is passed through a set of tubing, referred to as a filtration circuit, via a machine to a semi-permeable membrane (the filter) where waste products and water (collectively called ultrafiltrate) are removed by convection. For treatment of inflammation, including sepsis, the filter comprises the DAMP-binding polypeptide of SEQ ID NO: 1, or the OP18 polypeptide of SEQ ID NO: 2, for removal of DAMPs from the treated subject's blood through specific binding of DAMPs to the polypeptides. Replacement fluid and blood is then returned to the patient. Accordingly, the present disclosure provides hemofilters comprising the DAMP-binding polypeptide of SEQ ID NO: 1, or the OP18 polypeptide of SEQ ID NO: 2 bound to said hemofilter.

In one aspect, a diagnostic method for detection of inflammation in a subject is provided comprising the step of contacting a subject sample to a DAMP-binding polypeptide and detecting the binding of said DAMP-binding polypeptide to one of more DAMPs within the sample wherein an increase in binding of said DAMP-binding polypeptide to one of more DAMPs within the sample, compared to a control sample, indicates the presence of inflammation. In one non-limiting embodiment, the detection of binding of said DAMP-binding protein to one of more DAMPs within the sample is through an ELISA assay. In another embodiment, the diagnosed inflammation is sepsis.

In another aspect of the embodiment, an article of manufacture (e.g., a kit) containing materials useful for the treatment of inflammation-mediated disorders as described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition, e.g., inflammation such as sepsis and may have a sterile access port (for example the container may be an intravenous solution bag or a vial having a stopper pierceable by a hypodermic injection needle).

Kits in certain embodiments may comprise a package insert indicating that the compositions can be used to treat a particular condition, e.g. inflammation such as sepsis. Alternatively, or additionally, the kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one aspect, the article of manufacture may comprise (a) a first container with a composition contained therein, wherein the composition comprises DAMP-binding polypeptide, or OP18 polypeptide; and, optionally, (b) a second container with a composition contained therein, wherein the composition comprises a further therapeutic agent.

In yet another embodiment, kits comprising the DAMP-binding polypeptide of SEQ ID NO: 1, or the OP18 polypeptide of SEQ ID NO:2, may be used to detect DAMPs in a patient sample, thereby providing a method for diagnosis of inflammatory diseases. For example, said kit may contain reagents for performing an ELISA assay that employs the DAMP-binding polypeptide of SEQ ID NO: 1, or the OP18 polypeptide of SEQ ID NO:2, for detection of DAMPs present in a subject's sample. Demonstration of OP18's ability to bind and capture multiple DAMPs present in the blood of septic patients, provides a useful reagent for detecting multiple DAMPs simultaneously in clinical samples.

It is understood that the examples and embodiments described herein are for illustrative purposes only and that various modifications or changes in light thereof will be suggested to persons skilled in the art and are to be included within the spirit and purview of this application and scope of the appended claims. All documents, papers and published materials referenced herein, including books, journal articles, manuals, patent applications, published patent applications and patents, are expressly incorporated herein by reference in their entireties.

### Example

During sepsis, stressed cells release intracellular molecules that, when outside the cells, become pro-inflammatory mediators known as damage-associated molecular patterns (DAMPs) (Nofi C P, Wang P, Aziz M. Chromatin-Associated Molecular Patterns (CAMPs) in sepsis. Cell Death Dis. 2022; 13(8):700; Denning N L, Aziz M, Gurien S D, Wang P. DAMPs and NETs in Sepsis. Front Immunol. 2019; 10:2536). The studies described herein focused on DAMPs that originate from the nucleus, such as extracellular cold-inducible RNA-binding protein (eCIRP), high mobility group box 1 (HMGB1), and histone H3 (Nofi C P, Wang P, Aziz M. Chromatin-Associated Molecular Patterns (CAMPs) in sepsis. Cell Death Dis. 2022; 13(8):700). These DAMPs bind to Toll-like receptor 4 (TLR4), amplifying inflammation and contributing to organ injury and death in sepsis (Nofi C P, Wang P, Aziz M. Chromatin-Associated Molecular Patterns (CAMPs) in sepsis. Cell Death Dis. 2022; 13(8):700; Denning N L, Aziz M, Gurien S D, Wang P. DAMPs and NETs in Sepsis. Front Immunol. 2019; 10:2536).

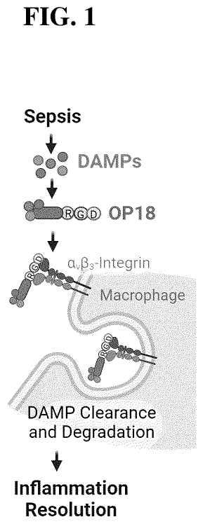

The novel compound referred to herein as Opsonic Peptide 18 (OP18) is demonstrated to clear multiple DAMPs. A 15-amino acid (aa) was identified that shared binding site at the extracellular domain of TLR4 for eCIRP, HMGB1, and histone H3. This 15-aa peptide was engineered by tagging it with an αᵥβ₃-integrin binding RGD (Arg-Gly-Asp) motif, resulting in the development of an 18-aa innovative multiple DAMP-scavenging peptide, OP18. OP18 is a highly innovative novel chimeric peptide that acts as a bridging molecule. It simultaneously recognizes multiple DAMPs and the αᵥβ₃-integrin of macrophages through its RGD sequence. This dual recognition facilitates the phagocytic clearance of DAMPs, thereby promoting the resolution of inflammation during sepsis. Thus, OP18 can simultaneously recognize multiple DAMPs and the αᵥβ₃-integrin, clearing DAMPs by the phagocytic cells (macrophages, neutrophils, B cells) and leading to resolution of inflammation. (FIG. 1)

The experimental data described herein was designed to elucidate OP18's mechanism in scavenging multiple DAMPs to regulate hyperimmune response. It also seeks to demonstrate correction of macrophage phagocytic dysfunction during inflammation by restoring metabolic homeostasis. The clearance of DAMPs triggers the release of the anti-inflammatory cytokine IL-10, which corrects abnormal metabolism, reverses epigenetic and transcriptomic changes, and regulates inflammation while restoring normal phagocytic function. Thus, it is believed that OP18 clears multiple nuclear DAMPs via αᵥβ₃-integrin, leading to metabolic reprogramming to reduce inflammation, correct macrophage phagocytic dysfunction, and improve overall survival in sepsis.

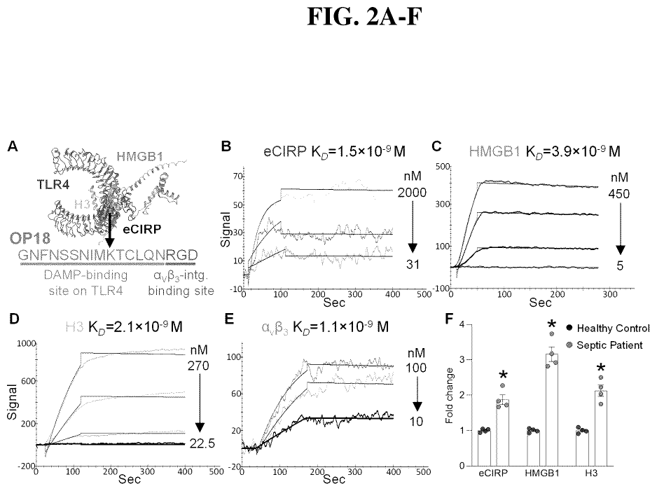

Using in silico model, a 15-aa binding region (aa234-248) at the extracellular domain of TLR4 shared by multiple DAMPs, such as eCIRP, HMGB1, and histone H3 was identified (FIG. 2A). It was confirmed that this 15-aa TLR4 region does not overlap with either the TLR4 dimer formation (aa342-531) or the MD2 binding site (aa24-47) of TLR4 indicating that a peptide containing this 15-aa motif is unlikely to interfere with TLR4's function and serve as a TLR4 antagonist (Sullivan C, Charette J, Catchen J, Lage C R, Giasson G, Postlethwait J H, et al. The gene history of zebrafish tlr4a and tlr4b is predictive of their divergent functions. J Immunol. 2009; 183(9):5896-908; Kim H M, Park B S, Kim J I, Kim S E, Lee J, Oh S C, et al. Crystal structure of the TLR4-MD-2 complex with bound endotoxin antagonist Eritoran. Cell. 2007; 130(5):906-17). Next, this 15-aa peptide was engineered by incorporating an RGD (Arginine-Glycine-Aspartate) domain to facilitate recognition by macrophages through αᵥβ₃-integrin, resulting in the invention of an 18-aa multi-DAMP-scavenging peptide, OP18 (GNFNSSNIMKTCLQNRGD) (SEQ ID NO: 2) (Kapp T G, Rechenmacher F, Neubauer S, Maltsev O V, Cavalcanti-Adam E A, Zarka R, et al. A Comprehensive Evaluation of the Activity and Selectivity Profile of Ligands for RGD-binding Integrins. Sci Rep. 2017; 7:39805; Pang X, He X, Qiu Z, Zhang H, Xie R, Liu Z, et al. Targeting integrin pathways: mechanisms and advances in therapy. Signal Transduct Target Ther. 2023; 8(1):1). The amino acid sequence homology between mouse and human CIRP is 97%, whereas that of HMGB1 and histone H3, along with the region of eCIRP that binds to TLR4, is 100%. This suggests that OP18 can equally recognize mouse and human eCIRP, HMGB1, and H3. A BIAcore (surface plasmon resonance) assay was performed to validate the quantitative binding affinity of OP18 with eCIRP, HMGB1, and H3, revealing antigen-antibody-like binding affinity (FIG. 2B-D). Importantly, the binding affinity of eCIRP and HMGB1 for OP18 is 10× greater than their affinity for TLR4, indicating that OP18 can divert and sequester these DAMPs (Qiang X, Yang W L, Wu R, Zhou M, Jacob A, Dong W, et al. Cold-inducible RNA-binding protein (CIRP) triggers inflammatory responses in hemorrhagic shock and sepsis. Nat Med. 2013; 19(11):1489-95; Yang H, Hreggvidsdottir H S, Palmblad K, Wang H, Ochani M, Li J, et al. A critical cysteine is required for HMGB1 binding to Toll-like receptor 4 and activation of macrophage cytokine release. Proc Natl Acad Sci USA. 2010; 107(26):11942-7). It has also been demonstrated that OP18 strongly binds to αᵥβ₃-integrin, indicating OP18's potential to simultaneously bind DAMPs and αᵥβ₃-integrin of phagocytes (FIG. 2E).

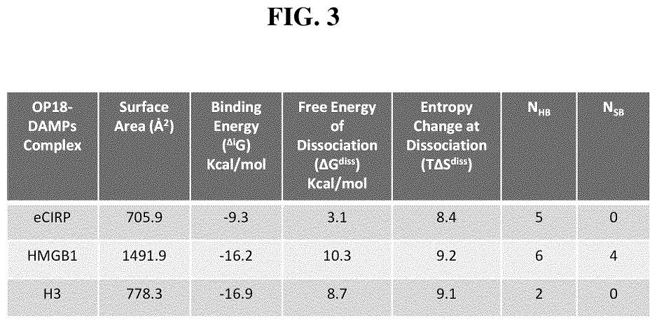

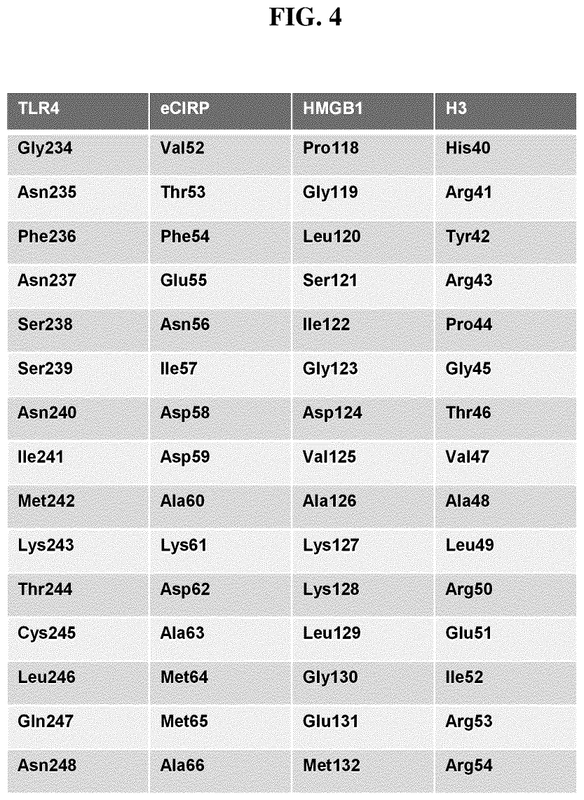

To explore OP18's potential clinical value, its remarkable ability to bind to eCIRP, HMGB1, and H3 in the blood of septic patients was demonstrated, indicating OP18's potential in clearing these DAMPs in septic patients (FIG. 2F). FIG. 3 demonstrates the quantitative binding interaction between OP18, and various DAMPs as revealed by the surface area, binding energy, free energy dissociation, and entropy change at dissociation. Through computational modeling, the putative amino acid sequence on TLR4 extracellular motif that recognize the sequences of various DAMPs (eCIRP, HMGB1, histone H3) has been determined (FIG. 4).

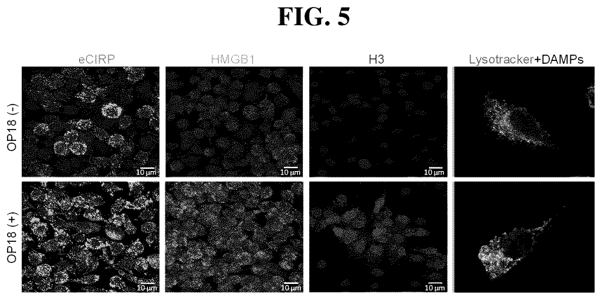

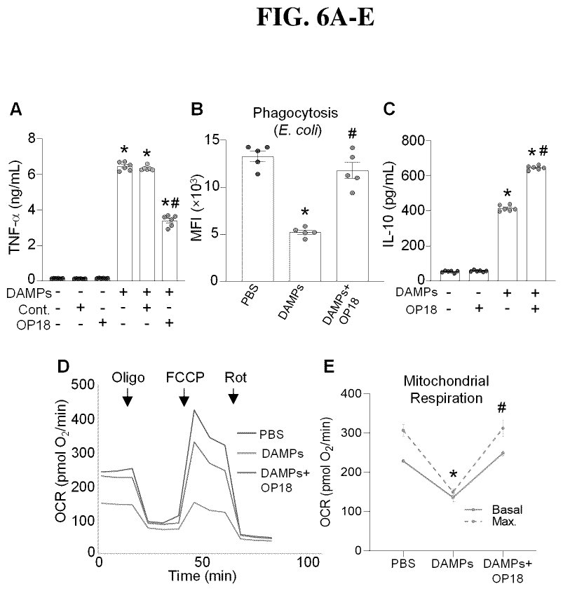

Next, it was revealed that OP18 markedly enhances the internalization of DAMPs by macrophages, leading to their fusion with lysosomes for subsequent degradation (FIG. 5). The clearance of DAMPs attenuates inflammation as OP18 significantly decreases macrophage release of TNFα (FIG. 6A). Macrophages treated with the scrambled peptide (NRKINMGSFTQCSNNLGD (SEQ ID NO: 3) as control) show no alterations in TNFα release, indicating OP18's specificity in targeting DAMPs. Controls were utilized in all studies and no changes in the results were observed compared to those treated with DAMPs. During inflammation, macrophages experience phagocytic dysfunction (Zhou M, Aziz M, Yen H T, Ma G, Murao A, Wang P. Extracellular CIRP dysregulates macrophage bacterial phagocytosis in sepsis. Cell Mol Immunol. 2023; 20(1):80-930; Hortová-Kohoutková M, Tidu F, De Zuani M, Šrámek V, Helán M, Frič J. Phagocytosis-Inflammation Crosstalk in Sepsis: New Avenues for Therapeutic Intervention. Shock. 2020; 54(5):606-140). The data described herein showed that OP18-mediated clearance of DAMPs corrects macrophage phagocytic dysfunction (FIG. 6B). It was also revealed that OP18 significantly increases IL-10 release from macrophages post-phagocytosis of DAMPs (FIG. 6C), aligning with prior finding that phagocytosis of apoptotic cells can promote IL-10 release (Elliott M R, Koster K M, Murphy P S. Efferocytosis Signaling in the Regulation of Macrophage Inflammatory Responses. J Immunol. 2017; 198(4):1387-94; Chung E Y, Liu J, Homma Y, Zhang Y, Brendolan A, Saggese M, et al. Interleukin-10 expression in macrophages during phagocytosis of apoptotic cells is mediated by homeodomain proteins Pbx1 and Prep-1. Immunity. 2007; 27(6):952-64; Boada-Romero E, Martinez J, Heckmann B L, Green D R. The clearance of dead cells by efferocytosis. Nat Rev Mol Cell Biol. 2020; 21(7):398-414; Greenlee-Wacker M C. Clearance of apoptotic neutrophils and resolution of inflammation. Immunol Rev. 2016; 273(1):357-70). IL-10 induces metabolic reprogramming in macrophages during inflammation (Ip W K E, Hoshi N, Shouval D S, Snapper S, Medzhitov R. Anti-inflammatory effect of IL-10 mediated by metabolic reprogramming of macrophages. Science. 2017; 356(6337):513-9). Notably, clearance of DAMPs by OP18 rectifies macrophage metabolic abnormalities by restoring mitochondrial activity (oxidative phosphorylation), as evidenced by improved oxygen consumption rate (OCR) (FIG. 6D, E). Metabolic restoration could ameliorate phagocytic dysfunction, as metabolic reprogramming is linked to enhanced phagocytic potential (Pavlou S, Wang L, Xu H, Chen M. Higher phagocytic activity of thioglycollate-elicited peritoneal macrophages is related to metabolic status of the cells. J Inflamm (Lond). 2017; 14:4; O'Neill L A, Kishton R J, Rathmell J. A guide to immunometabolism for immunologists. Nat Rev Immunol. 2016; 16 (9): 553-65; Gauthier T, Chen W. Modulation of Macrophage Immunometabolism: A New Approach to Fight Infections. Front Immunol. 2022; 13:780839).

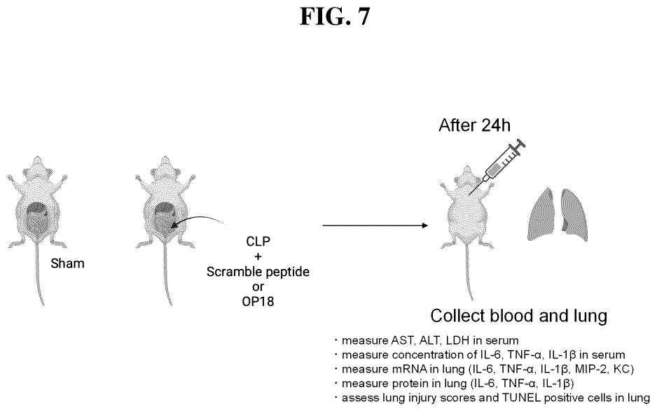

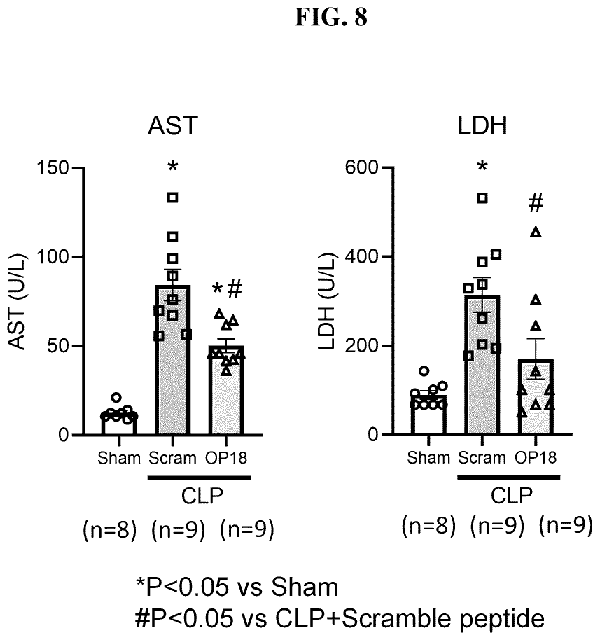

The therapeutic potential of OP18 in sepsis due to intestinal perforation was further investigated using the cecal ligation and puncture (CLP) model (FIG. 7). In this regard, C57BL/6 mice were induced sepsis by cecal ligation and puncture (CLP). These mice were simultaneously treated with either OP18 (0.2 mg/kg body weight) or the same dose of scrambled peptide via intraperitoneal (i.p.) injection. After 24 h of CLP and/or OP18 and scrambled peptide injection, blood and lungs were collected to perform various tests. It was found that treatment of septic mice with OP18 significantly decreases systemic tissue injury markers, such as aspartate aminotransferase (AST) and lactate dehydrogenase (LDH) levels compared to scrambled-treated septic mice (FIG. 8).

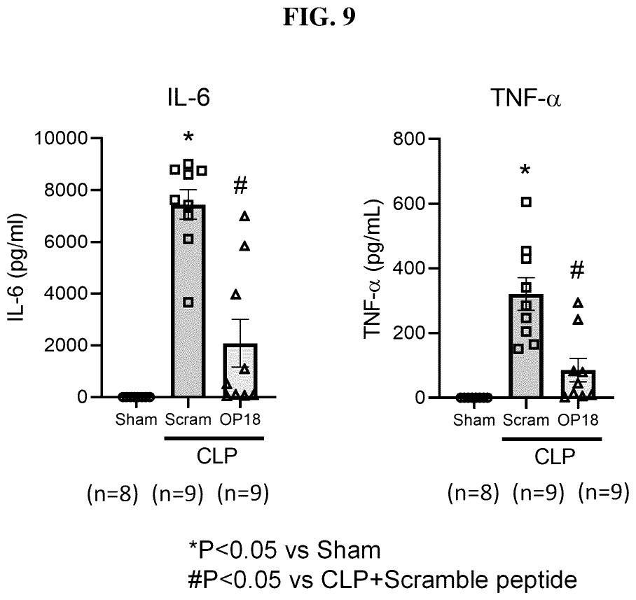

It has also been revealed that treatment of septic mice with OP18 significantly decreases serum levels of pro-inflammatory cytokines, such as interleukin-6 (IL-6) and tumor necrosis factor-α (TNF-α) compared to scrambled-treated septic mice (FIG. 9).

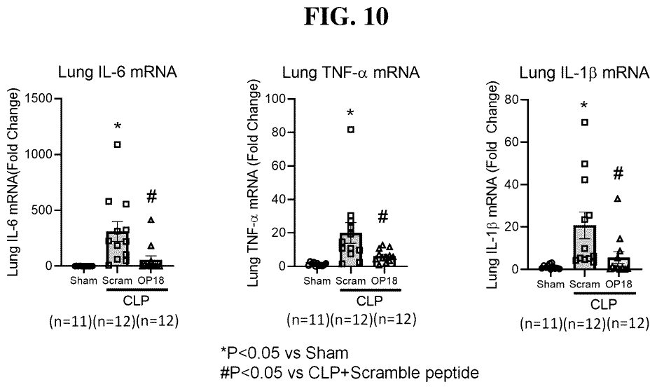

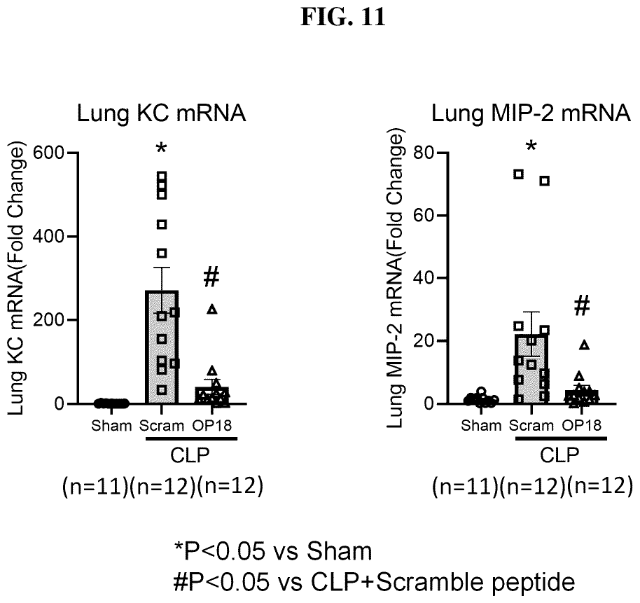

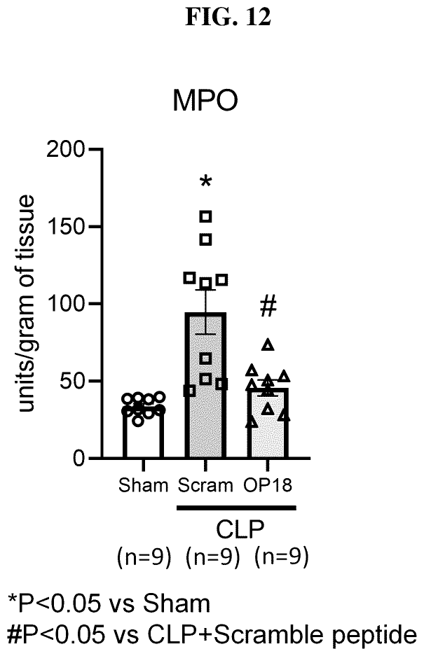

OP18 treatment in septic mice has also been shown to significantly decrease lung IL-6, TNF-α, and IL-1β expression at mRNA level as compared to scrambled peptide treated mice (FIG. 10). A significant decrease of the lung chemokine (KC and MIP-2) mRNA expression (FIG. 11) and activity of lung myeloperoxidase (MPO), a marker of neutrophil infiltration/granulation in OP18-treated mice compared to scrambled peptide-treated mice with sepsis (FIG. 12) was found.

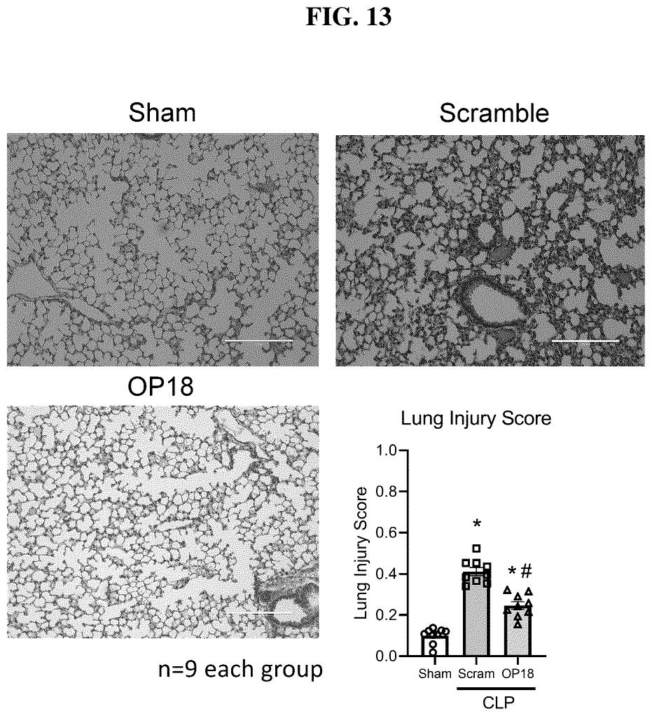

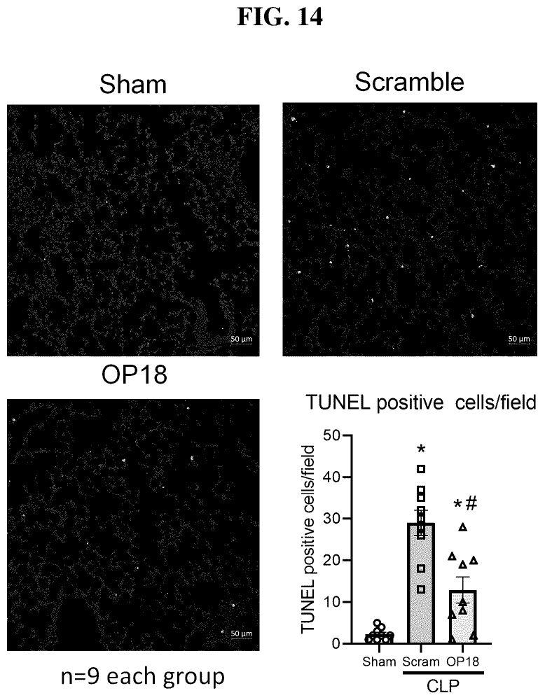

It was also demonstrated that treatment of septic mice with OP18 significantly decreased lung injury score (FIG. 13) and decreased cellular apoptosis (FIG. 14) in the lungs, compared to scrambled peptide treated conditions.

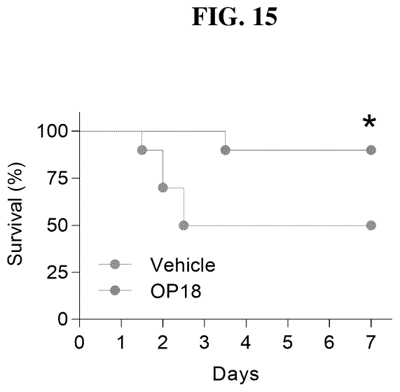

Finally, a survival assay was performed to determine the overall survival impact of OP18-treated mice in sepsis. The data showed that treatment of septic mice with OP18 significantly improved overall survival rate compared to vehicle (PBS)-treated septic mice (FIG. 15).

What is claimed is:

1. An OP18 polypeptide having at least about 90% or more identity with the polypeptide of SEQ ID NO:2.

2. The OP18 polypeptide of claim 1 comprising SEQ ID NO: 2.

3. The OP18 polypeptide of claim 1, wherein said polypeptide comprises modifications to increase stability and efficacy of the polypeptide.

4. The OP18 polypeptide of claim 1, associated with a nanoparticle.

5. A nucleic acid encoding the OP18 polypeptide of claim 1.

6. A cell comprising the nucleic acid of claim 5.

7. A pharmaceutical composition comprising the OP18 polypeptide of claim 1, and a pharmaceutically acceptable carrier.

8. A method of treating an inflammation-mediated disorder in a subject in need thereof comprising the administration of the pharmaceutical composition of claim 7 to said subject.

9. A DAMP-binding polypeptide, having at least about 90% or more homology with the polypeptide of SEQ ID NO:1.

10. The DAMP-binding polypeptide of claim 9, wherein said polypeptide comprises modifications to increase stability of the polypeptide.

11. A fusion polypeptide comprising (i) the DAMP-binding polypeptide of claim 9; and (2) a scavenger cell binding polypeptide.

12. The DAMP-binding polypeptide of claim 9, wherein the polypeptide is associated with a nanoparticle.

13. A nucleic acid encoding the DAMP-binding polypeptide of claim 9.

14. A nucleic acid encoding the fusion polypeptide of claim 11.

15. A pharmaceutical composition comprising the DAMP-binding polypeptide of claim 9, and a pharmaceutically acceptable carrier.

16. A pharmaceutical composition comprising the fusion polypeptide of claim 11.

17. A method of treating an inflammation-mediated disorder in a subject in need thereof comprising the administration of the pharmaceutical composition of claim 15 to said subject.

18. A method of treating an inflammation-mediated disorder in a subject in need thereof comprising the administration of the pharmaceutical composition of claim 16 to said subject.

19. A kit comprising the OP18 polypeptide of claim 1.

20. A diagnostic method for detection of inflammation in a subject, comprising the step of contacting a subject sample to the OP-18 polypeptide of claim 1 and detecting the binding of said OP-18 polypeptide to one of more DAMPs within the sample wherein an increase in binding of said OP-18 polypeptide, to one or more DAMPs, compared to a control sample, indicates the presence of inflammation.
